(19) United States
(12) Patent Application Publication
(10) Pub. No.: US 2026/0344989 A1
(43) Pub. Date: Nov. 5, 2026

(54) ENDOSCOPE MULTI-LUMEN TUBE AND ENDOSCOPE

(71) Applicant: OLYMPUS MEDICAL SYSTEMS CORP., Tokyo (JP)

(72) Inventor: Takuto YOSHINAGA, Hino-shi (JP)

(73) Assignee: OLYMPUS MEDICAL SYSTEMS CORP., Tokyo (JP)

(21) Appl. No.: 19/712,150

(22) Filed: Jun. 18, 2026

Related U.S. Application Data
(63) Continuation of application No. PCT/JP2024/040801, filed on Nov. 18, 2024.
(60) Provisional application No. 63/550,634, filed on Feb. 7, 2024.

(51) Int. Cl. A61B 1/018; A61B 1/015; A61M 25/00

(52) U.S. Cl. CPC A61B 1/018; A61B 1/015; A61M 2025/0035; A61M 2025/0037

(57) ABSTRACT

An endoscope multi-lumen tube 11 according to the present disclosure includes a tube 12 arranged within an insertion portion 2 of an endoscope 1 in a longitudinal direction; a deformable wall 13 partitioning an interior of the tube; and a first channel 5 and a second channel 7 formed on one portion and another portion, respectively, of the interior of the tube partitioned by the wall. In a cross-section perpendicular to the longitudinal direction, the tube is configured such that: a side of the first channel protrudes toward one side relative to the wall; a side of the second channel protrudes toward another side relative to the wall; and outer contours 12a1 and 12a2 of two parts connected to the wall each form a recessed shape.

### CROSS REFERENCE TO RELATED APPLICATION

This application is a continuation application of PCT/JP2024/040801 filed on Nov. 18, 2024, which claims the benefit of U.S. Provisional Patent Application No. 63/550,634, filed on Feb. 7, 2024. The entire disclosures of the above-referenced applications are incorporated by reference herein.

### FIELD

The present disclosure relates to an endoscope multi-lumen tube wherein a plurality of channels are formed in the interior of a tube, and to an endoscope including the endoscope multi-lumen tube.

### BACKGROUND

Endoscopes are used to observe the inside of a body cavity of a subject. Some types of endoscopes include both a liquid feeding channel (or a gas/liquid feeding channel that also serves as a gas feeding channel) and a treatment instrument channel.

For example, Japanese Patent Publication No. 5,305,859 describes an endoscope including a gas/liquid conduit and a forceps conduit. A gas/liquid feeding channel is provided within the gas/liquid conduit, and a treatment instrument channel is provided within the forceps conduit (treatment instrument conduit).

### SUMMARY

An endoscope multi-lumen tube according to one aspect of the present disclosure includes a tube arranged within an insertion portion of an endoscope in a longitudinal direction; a deformable wall provided along the longitudinal direction and partitioning an interior of the tube; a first channel formed on one portion of the interior of the tube partitioned by the wall; and a second channel formed on another portion of the interior of the tube partitioned by the wall. In a cross-section perpendicular to the longitudinal direction, the tube is configured such that: a side of the first channel protrudes toward one side relative to the wall; a side of the second channel protrudes toward another side relative to the wall; and outer contours of two parts connected to the wall each form a recessed shape.

An endoscope according to one aspect of the present disclosure includes an insertion portion; and an endoscope multi-lumen tube. The endoscope multi-lumen tube includes a tube arranged within the insertion portion of an endoscope in a longitudinal direction; a deformable wall provided along the longitudinal direction and partitioning an interior of the tube; a first channel formed on one portion of the interior of the tube partitioned by the wall; and a second channel formed on another portion of the interior of the tube partitioned by the wall. In a cross-section perpendicular to the longitudinal direction, the tube is configured such that: a side of the first channel protrudes toward one side relative to the wall; a side of the second channel protrudes toward another side relative to the wall; and outer contours of two parts connected to the wall each form a recessed shape.

### BRIEF DESCRIPTION OF DRAWINGS

FIG. 1 is a view showing a configuration of an endoscope according to a first embodiment of the present disclosure.

FIG. 2 is a view showing a configuration of an insertion portion of the endoscope according to the first embodiment.

FIG. 3 is a cross-sectional view taken along line III-III in FIG. 2, perpendicular to a longitudinal direction of the insertion portion of the endoscope in the first embodiment.

FIG. 4 is a cross-sectional view taken along line IV-IV in FIG. 2, perpendicular to the longitudinal direction of the insertion portion of the endoscope in the first embodiment.

FIG. 5 is a diagram illustrating an operation in which a first wall and a second wall partitioning the interior of a tube deform, in an endoscope multi-lumen tube of the first embodiment.

FIG. 6 is a cross-sectional view of the endoscope multi-lumen tube taken perpendicular to the longitudinal direction, showing an example configuration that restricts the amount of protrusion of the walls toward the side of a treatment instrument channel in the first embodiment.

FIG. 7 is a cross-sectional view of the endoscope multi-lumen tube taken perpendicular to the longitudinal direction, showing an example configuration that restricts the amount of protrusion of the walls toward the side of a liquid feeding channel in the first embodiment.

FIG. 8 is a flowchart showing a reprocessing method for an endoscope in the first embodiment.

FIG. 9 is a diagram for illustrating a configuration and operation of a first wall and a second wall partitioning the interior of a tube in an endoscope multi-lumen tube of a second embodiment of the present disclosure.

FIG. 10 is a view showing a configuration of an endoscope of a related art.

FIG. 11 is a cross-sectional view of the endoscope of the related art taken along line XI-XI shown in FIG. 10.

FIG. 12 is a cross-sectional view of an insertion portion of an endoscope in a first modified example of the related art, taken perpendicular to the longitudinal direction.

FIG. 13 is a view showing arrangement of a treatment instrument channel tube in an insertion portion of an endoscope in a second modified example of the related art.

FIG. 14 is a cross-sectional view of an insertion portion of an endoscope in a third modified example of the related art, taken perpendicular to the longitudinal direction.

### DESCRIPTION OF EMBODIMENTS

An embodiment of the present disclosure will now be described with reference to the drawings. However, the present disclosure is not limited to the embodiments described below.

Note that in the drawings, identical or corresponding elements are assigned the same reference numerals as appropriate. The drawings are schematic, and the relationship between the lengths of each element, the ratio of the lengths of each element, the quantity of each element, and the like, in one drawing may differ from reality in order to simplify the explanation. Furthermore, the drawings may contain parts in which length relationships, ratios, quantities, and the like are different from each other.

### First Embodiment

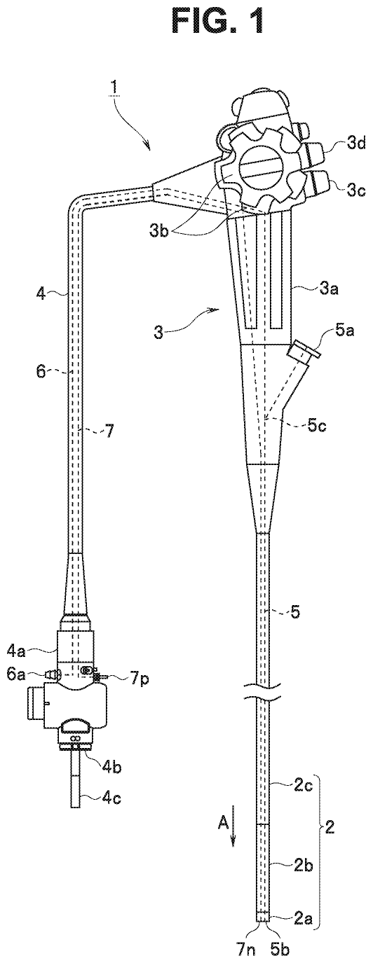

FIGS. 1 to 8 illustrate a first embodiment of the present disclosure. FIG. 1 is a view showing a configuration of an endoscope 1 of the first embodiment.

The endoscope 1 is an insertion instrument including a section to be inserted into a subject. The subject may be either a living body, such as a human or animal, or a non-living body, such as a machine or a building.

The endoscope 1 includes an insertion portion 2, an operation portion 3, and a universal cable 4.

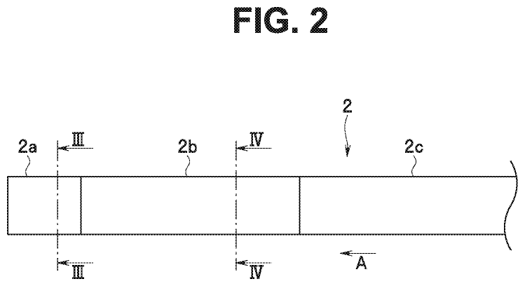

FIG. 2 is a view showing configurations of the insertion portion 2 of the endoscope 1 of the first embodiment.

The insertion portion 2 is a section configured to be inserted into the subject. The insertion portion 2 includes, sequentially from a distal end side toward a proximal end side, a distal end portion 2a, a bending portion 2b, and a flexible tube portion 2c. It should be noted that the direction from the distal end side toward the proximal end side of the insertion portion 2 is opposite to a longitudinal direction A of the insertion portion 2, indicated by an arrow in FIGS. 1 and 2, etc.

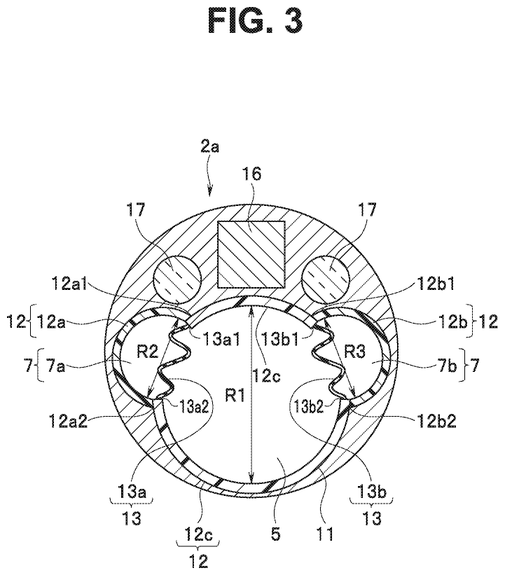

In the distal end portion 2a, an image pickup module 16 (see, for example, FIG. 3), an illumination optical system, a distal end side opening 5b of the treatment instrument channel, the nozzle 7n, and the like are arranged.

The illumination optical system irradiates the subject with illumination light transmitted by the light guide 17 (see, for example, FIG. 3).

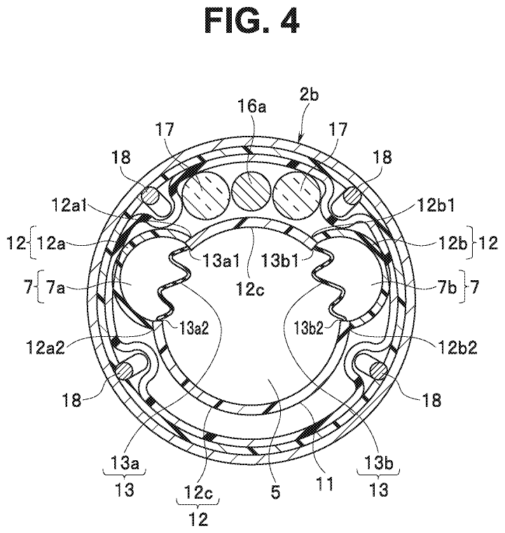

The image pickup module 16 includes an image pickup device and an image pickup system. The image pickup system forms an optical image of the subject. The image pickup device generates an image pickup signal by photoelectrically converting the optical image formed by the image pickup system. The image pickup module 16 is connected to a signal cable 16a (see, for example, FIG. 4).

The distal end side opening 5b of the treatment instrument channel communicates with the treatment instrument channel 5 (first channel), and is an opening on the distal end side of the treatment instrument channel 5.

The nozzle 7n communicates with a liquid feeding channel 7 (second channel, third channel). The nozzle 7n is arranged toward an observation window at a distal end of the image pickup system, and discharges fluid (assumed to be liquid, but may include gas) fed via the liquid feeding channel 7 toward the observation window at the distal end of the image pickup system. It should be noted that here, an example is described where the distal end side opening of the liquid feeding channel 7 is the nozzle 7n, but the distal end side opening is not limited to this configuration. The distal end side opening of the liquid feeding channel 7 may also be configured for forward liquid feeding (liquid feeding toward the front direction and/or observation direction).

The bending portion 2b is, for example, a section capable of bending in four directions: up/down and left/right directions.

The flexible tube portion 2c is a tube portion having flexibility. It should be noted that here an example is given where the endoscope 1 is a flexible endoscope including the flexible tube portion 2c. However, the endoscope 1 may also be a rigid endoscope in which the part corresponding to the flexible tube portion 2c is rigid in form.

The operation portion 3 is disposed on the proximal end side of the insertion portion 2. The operation portion 3 is a section for the user to operate the endoscope 1. The operation portion 3 includes a grasping portion 3a, a bending operation knob 3b, a liquid feeding button 3c, a suction button 3d, and a treatment instrument insertion port 5a.

The grasping portion 3a is a section where the user grasps the endoscope 1 with the palm.

The bending operation knob 3b is an operating device for controlling the bending of the bending portion 2b. The bending operation knob 3b includes an up/down bending operation knob and a left/right bending operation knob. Operating the bending operation knob 3b pulls four bending operation wires 18 (see, for example, FIG. 4), causing the bending portion 2b to bend.

When the bending portion 2b is bent, the direction of the distal end portion 2a changes. Consequently, the image pickup direction of the image pickup module 16 and the irradiation direction of the illumination light of the illumination optical system change. The bending portion 2b is also bent to improve the insertability of the insertion portion 2 within the subject.

The liquid feeding button 3c is a button for operating liquid feeding to the observation window of the distal end portion 2a. Operating the liquid feeding button 3c injects fluid into the liquid feeding channel 7, causing liquid to be fed from the nozzle 7n. The fluid fed from the nozzle 7n cleans the observation window. It should be noted that while the present embodiment gives an example of providing the endoscope 1 with the liquid feeding channel 7, the endoscope 1 may be provided with a gas/liquid feeding channel capable of feeding both liquid and gas.

The treatment instrument insertion port 5a is an opening on the proximal end side of the treatment instrument channel 5. The treatment instrument channel 5 is arranged from the distal end side opening 5b of the treatment instrument channel at the distal end portion 2a of the insertion portion 2 to the treatment instrument insertion port 5a of the operation portion 3.

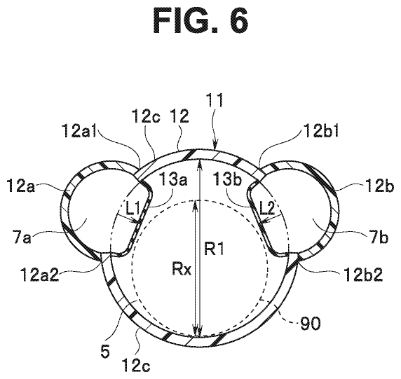

A treatment instrument 90 (see FIG. 6), such as forceps, is inserted into the treatment instrument channel 5 from the treatment instrument insertion port 5a. A distal end portion of the treatment instrument 90 is guided from the treatment instrument channel 5 to the distal end side opening 5b of the treatment instrument channel, and protrudes into the subject. Various treatments are performed on the subject using the protruding distal end portion of the treatment instrument 90.

The suction button 3d is a button for performing an operation of suctioning inside the subject from the distal end portion 2a via a suction channel 6. The suction channel 6 connects to the treatment instrument channel 5 at a branch point 5c and communicates with the distal end side opening 5b of the treatment instrument channel. Therefore, the treatment instrument channel 5 from the branch point 5c to the distal end side opening 5b of the treatment instrument channel also serves to function as the suction channel 6.

Thus, suction from within the subject is performed by the suction channel 6 via the treatment instrument channel 5 from the distal end side opening 5b of the treatment instrument channel to the branch point 5c. When the suction operation is performed, for example, liquid, gas, mucus or the like is suctioned from within the subject.

The universal cable 4 is a composite cable incorporating the signal cable 16a, the light guide 17, the suction channel 6, the liquid feeding channel 7, and the like. The universal cable 4 extends from, for example, a side surface on the proximal end side of the operation portion 3. A connector 4a is provided at an extension end of the universal cable 4. The connector 4a connects the endoscope 1 to an endoscope processor (video processor), a light source apparatus, a suction pump, a liquid feeding pump, and a liquid feeding tank, or the like.

The connector 4a is provided with, for example, an electrical contact 4b, a light guide connector 4c, a suction ferrule 6a, and a liquid feeding ferrule 7p.

The electrical contact 4b transmits signals sent via the signal cable 16a between the endoscope 1 and the endoscope processor.

The light guide connector 4c transmits illumination light generated by the light source apparatus to the light guide 17.

The suction ferrule 6a communicates with the suction channel 6. The suction channel 6 is arranged from the suction ferrule 6a to the branch point 5c of the operation portion 3. The suction ferrule 6a is connected to a suction pump.

The liquid feeding ferrule 7p communicates with the liquid feeding channel 7. The liquid feeding channel 7 is arranged from the liquid feeding ferrule 7p to the nozzle 7n of the distal end portion 2a of the insertion portion 2. The liquid feeding ferrule 7p is connected to the liquid feeding pump and the liquid feeding tank.

FIG. 3 is a cross-sectional view taken along line III-III in FIG. 2, perpendicular to the longitudinal direction A of the insertion portion 2 of the endoscope 1 in the first embodiment.

The distal end portion 2a is provided with an endoscope multi-lumen tube 11, the image pickup module 16, and two light guides 17.

FIG. 4 is a cross-sectional view taken along line IV-IV in FIG. 2, perpendicular to the longitudinal direction A of the insertion portion 2 of the endoscope 1 in the first embodiment.

The bending portion 2b is provided with the endoscope multi-lumen tube 11, the signal cable 16a, the two light guides 17, and four bending operation wires 18. Distal ends of the four bending operation wires 18 are fixed to the proximal end side of the distal end portion 2a or the distal end side of the bending portion 2b.

As shown in FIGS. 3 and 4, the endoscope multi-lumen tube 11 includes a tube 12, and walls 13 partitioning the interior of the tube 12.

One channel is formed on one side of the interior of the tube 12 partitioned by the walls 13, and another channel is formed on the other side of the interior. That is, by partitioning the interior of the tube 12 with the walls 13 along the longitudinal direction A, the endoscope multi-lumen tube 11 including a plurality of channels is configured. The plurality of channels formed in the interior of the tube 12 will be described in detail below.

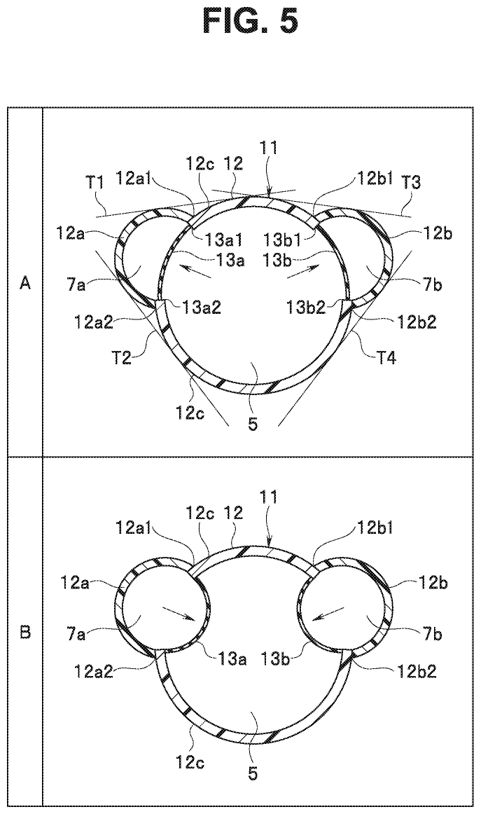

The tube 12 is arranged within the insertion portion 2 of the endoscope 1 along the longitudinal direction A. The walls 13 are provided in the interior of the tube 12 along the longitudinal direction A. As will be described later with reference to FIG. 5, etc., the walls 13 are flexible and configured to be deformable.

In the example shown in FIGS. 3 to 7, the liquid feeding channel 7 includes two channels; a first liquid feeding channel 7a and a second liquid feeding channel 7b. Such a configuration including a plurality of liquid feeding channels 7 is sometimes adopted, for example, in choledochoscopes. Choledochoscopes are known, among a plurality of types of gastrointestinal endoscopes, for having a small diameter of the insertion portion 2.

The following describes an example where the endoscope 1 includes two liquid feeding channels 7 according to the drawings, but the number of the liquid feeding channels 7 that the endoscope 1 includes may be one, or three or more, as needed.

The walls 13 include a first wall 13a and a second wall 13b. The first wall 13a and the second wall 13b are provided at different locations in the interior of the tube 12.

The first wall 13a partitions and watertightly separates the treatment instrument channel 5 (first channel) from the first liquid feeding channel 7a (second channel). The second wall 13b partitions and watertightly separates the treatment instrument channel 5 (first channel) from the second liquid feeding channel 7b (third channel).

As shown in FIG. 3, a distance between both ends 13a1 and 13a2 of the first wall 13a is smaller than a maximum diameter R1 of the treatment instrument channel 5. Furthermore, it may be preferable that the distance between the both ends 13a1 and 13a2 of the first wall 13a be smaller than a maximum diameter R2 of the first liquid feeding channel 7a.

A distance between both ends 13b1 and 13b2 of the second wall 13b is smaller than the maximum diameter R1 of the treatment instrument channel 5. Furthermore, it may be preferable that the distance between the both ends 13b1 and 13b2 of the second wall 13b be smaller than a maximum diameter R3 of the second liquid feeding channel 7b.

The walls 13 (first wall 13a and second wall 13b) are flexible and deformable (deformable in shape particularly in a cross-section perpendicular to the longitudinal direction A). For example, the tube 12 is configured of a material having a Shore hardness of 55 or higher, and the walls 13 are configured of a material having a Shore hardness of 30 or less.

The Shore hardness of a material configuring the tube 12 may be equal to or more than 55 and is equal to or less than 75. The Shore hardness of a material configuring the walls 13 may be 10 or more and 30 or less. To give a specific numerical example, the tube 12 is configured of a material having a Shore hardness of 65, and the walls 13 are configured of a material having a Shore hardness of 10.

The tube 12 includes a first part 12a configuring an outer wall of the first liquid feeding channel 7a, a second part 12b configuring an outer wall of the second liquid feeding channel 7b, and a third part 12c configuring an outer wall of the treatment instrument channel 5.

The tube 12 is configured such that, in a cross-section perpendicular to the longitudinal direction A, the third part 12c on the side of the treatment instrument channel 5 protrudes toward one side relative to the first wall 13a, and the first part 12a on the side of the first liquid feeding channel 7a protrudes toward the other side relative to the first wall 13a.

Furthermore, the tube 12 is configured such that the second part 12b on the side of the second liquid feeding channel 7b protrudes toward a different side from the third part 12c on the side of the treatment instrument channel 5 relative to the second wall 13b.

In a cross-section perpendicular to the longitudinal direction A, the first part 12a forms an arc with a diameter R2 (which is equal to the maximum diameter of the first liquid feeding channel 7a), the second part 12b forms an arc with a diameter R3 (which is equal to the maximum diameter of the second liquid feeding channel 7b), and the third part 12c forms an arc with a diameter R1 (which is equal to the maximum diameter of the treatment instrument channel 5).

The first part 12a is connected to the third part 12c at two sites at both ends of the first part 12a forming the arc. The second part 12b is connected to the third part 12c at two sites at both ends of the second part 12b forming the arc.

The both ends 13a1 and 13a2 of the first wall 13a are connected to the tube 12 at two sites of connection parts with the first part 12a and the third part 12c. The tube 12 is configured such that outer contours 12a1 and 12a2 of two parts connected to the both ends 13a1 and 13a2 of the first wall 13a each form a recessed shape. Specifically, for example, as shown in column A of FIG. 5 mentioned below, the outer contours 12a1 and 12a2 are formed in a recessed shape with respect to two common tangents T1 and T2 of the first part 12a and the third part 12c.

The both ends 13b1 and 13b2 of the second wall 13b are connected to the tube 12 at two sites of connection parts with the second part 12b and the third part 12c. The tube 12 is configured such that second outer contours 12b1 and 12b2 of two parts connected to the both ends 13b1 and 13b2 of the second wall 13b each form a recessed shape. Specifically, for example, as shown in column A of FIG. 5 mentioned below, the second outer contours 12b1 and 12b2 are formed in a recessed shape with respect to common tangents T3 and T4 of the second part 12b and the third part 12c.

With the outer contours 12a1 and 12a2 and the second outer contours 12b1 and 12b2, each forming a recessed shape, the tube 12 can prevent internal components other than the tube 12 within the insertion portion 2 from being displaced within the insertion portion 2.

As shown in FIGS. 3 and 4, one of the two light guides 17 is positioned in the vicinity of the outer contour 12a1 forming a recessed shape, and is disposed between the image pickup module 16 or the signal cable 16a and the first part 12a. The other of the two light guides 17 is positioned in the vicinity of the second outer contour 12b1 forming a recessed shape, and is disposed between the image pickup module 16 or the signal cable 16a and the second part 12b. Therefore, both of the two light guides 17 are prevented from displacement within the insertion portion 2 when bending is performed.

Here, if, for example, the light guide 17 undergoes displacement and is in a state such as intersecting with other internal components, effects may be caused such as a decline in the amount of illumination light transmitted by the light guide 17.

In contrast, in the present embodiment, the tube 12 is provided with the outer contours 12a1 and 12a2 and the second outer contours 12b1 and 12b2, each forming a recessed shape, with the light guides 17 arranged in the vicinity thereof. This prevents displacement of the light guides 17 and preemptively avoids a decline in the amount of illumination light.

Furthermore, even when internal components other than the light guide 17 are arranged in the vicinity of the outer contours 12a1 and 12a2 and the second outer contours 12b1 and 12b2, each forming a recessed shape, the effect of preventing displacement of the internal components due to the bending can similarly be obtained.

The provision of the outer contours 12a1 and 12a2 and the second outer contours 12b1 and 12b2, each forming a recessed shape, facilitates to arrange the internal components while preventing displacement thereof during assembly of the endoscope 1.

FIG. 5 is a diagram illustrating an operation in which the first wall 13a and the second wall 13b partitioning the interior of the tube 12 deform, in the endoscope multi-lumen tube 11 of the first embodiment.

Column A of FIG. 5 shows how the first wall 13a and the second wall 13b change when the treatment instrument 90 is inserted through the treatment instrument channel 5.

When the treatment instrument 90 is inserted through the treatment instrument channel 5 and presses against the first wall 13a and the second wall 13b, the walls protrude toward the sides of the first liquid feeding channel 7a and the second liquid feeding channel 7b, respectively.

Column B of FIG. 5 shows how the first wall 13a and the second wall 13b change when fluid is injected into the first liquid feeding channel 7a and the second liquid feeding channel 7b.

When fluid is injected into the first liquid feeding channel 7a and the second liquid feeding channel 7b, the first wall 13a and the second wall 13b each protrude toward the side of the treatment instrument channel 5 by the pressure of the fluid.

It should be noted that the treatment instrument 90 used for a flexible endoscope may be flexible. In this case, even when the treatment instrument 90 is inserted into the treatment instrument channel 5, the pressure received from the flexible treatment instrument 90 may make it difficult for the first wall 13a and the second wall 13b to protrude toward the sides of the first liquid feeding channel 7a and the second liquid feeding channel 7b.

Therefore, the fluid within the first liquid feeding channel 7a and the second liquid feeding channel 7b may be suctioned after stopping injection of the fluid into the first liquid feeding channel 7a and the second liquid feeding channel 7b. When the fluid is suctioned, the negative pressure of the suctioned fluid causes the first wall 13a and the second wall 13b to protrude toward the sides of the first liquid feeding channel 7a and the second liquid feeding channel 7b, respectively, thereby expanding the diameter of the treatment instrument channel 5.

Incidentally, in the endoscope 1, the use of the treatment instrument 90 and liquid feeding may be performed simultaneously. For example, when stones within the subject are crushed using a probe for lithotripsy, the field of view of the endoscope 1 may be obstructed by the crushed stones. In this case, fluid is injected into the first liquid feeding channel 7a and the second liquid feeding channel 7b in a state where the treatment instrument 90 is inserted through the treatment instrument channel 5, to secure the field of view.

At this time, if the difference between an inner diameter of the treatment instrument channel 5 and an outer diameter Rx of the treatment instrument 90 (see FIG. 6) is small, the first wall 13a and the second wall 13b may abut the treatment instrument 90 during liquid feeding. Consequently, after the walls abut the treatment instrument 90, the diameters of the first liquid feeding channel 7a and the second liquid feeding channel 7b may not expand, potentially causing the amount of liquid feeding to decline compared to when the treatment instrument 90 is not inserted through the treatment instrument channel 5.

Therefore, a configuration may be adopted that restricts the amount of deformation of the first wall 13a and the second wall 13b so that the first wall 13a and the second wall 13b do not interfere with the treatment instrument 90 during liquid feeding.

FIG. 6 is a cross-sectional view of the endoscope multi-lumen tube 11 taken perpendicular to the longitudinal direction A, showing an example configuration that restricts the amount of protrusion of the walls 13 toward the side of a treatment instrument channel 5 in the first embodiment.

For example, when the inner diameter (maximum diameter R1) of the treatment instrument channel 5 is approximately 2.0 mm and the outer diameter Rx of the treatment instrument 90 (such as a probe for lithotripsy) is approximately 1.5 mm, if the walls 13 protrude by 25% or more of the maximum diameter R1, they interfere with the treatment instrument 90.

Therefore, the walls 13 need only be configured such that, when fluid is injected into the liquid feeding channel 7, the maximum protrusion length of the walls 13 toward the side of the treatment instrument channel 5 is less than 25% of the maximum diameter R1 of the treatment instrument channel 5.

For example, consider a case where the walls 13 include the first wall 13a and the second wall 13b as mentioned above. The maximum protrusion length of the first wall 13a toward the side of the treatment instrument channel 5 when fluid is injected into the first liquid feeding channel 7a is defined as L1. L1 is the maximum protrusion length of the first wall 13a from a line corresponding to the inner diameter side of the third part 12c, indicated by a double-dash-dot line, toward the center of the treatment instrument channel 5.

The maximum protrusion length of the second wall 13b toward the side of the treatment instrument channel 5 when fluid is injected into the second liquid feeding channel 7b is defined as L2. L2 is the maximum protrusion length of the second wall 13b from a line corresponding to the inner diameter side of the third part 12c, indicated by a double-dash-dot line, toward the center of the treatment instrument channel 5.

Then, the first wall 13a and the second wall 13b need only be configured such that a sum (L1+L2) of the maximum protrusion length L1 and the maximum protrusion length L2 is less than 25% of the maximum diameter R1 of the treatment instrument channel 5.

It should be noted that less than 25% of the maximum diameter R1 is given as a numerical example, but the maximum protrusion length of the walls 13 may be restricted to a different value depending on the outer diameter Rx of the treatment instrument 90 assumed to be used.

By configuring the walls 13 (first wall 13a and second wall 13b) in this manner, an unintended decline in the amount of liquid feeding due to contact between the treatment instrument 90 and the walls 13 can be avoided.

It should be noted that a large-diameter biopsy treatment instrument can be given as an example of a large-diameter treatment instrument 90 that could potentially contact the walls 13. When using a biopsy treatment instrument, since the required amount of liquid feeding is not particularly high, a decline in the amount of liquid feeding may not pose a significant problem in some cases.

The walls 13 may not only restrict the amount of protrusion toward the side of the treatment instrument channel 5, but also restrict the amount of protrusion toward the side of the liquid feeding channel 7.

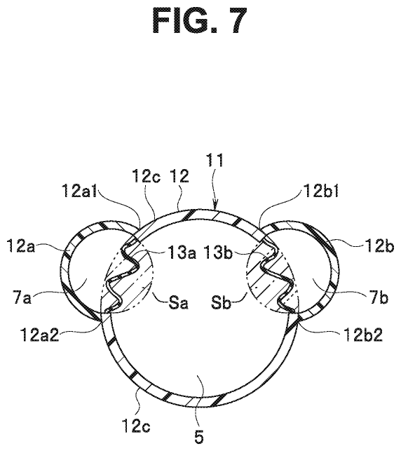

FIG. 7 is a cross-sectional view of the endoscope multi-lumen tube 11 taken perpendicular to the longitudinal direction A, showing an example configuration that restricts the amount of protrusion of the walls 13 toward the side of the liquid feeding channel 7 in the first embodiment.

An area Sa indicates the amount of change in cross-sectional area of the first liquid feeding channel 7a between a state where the first liquid feeding channel 7a enclosed by the first part 12a and the first wall 13a has a circular shape, and a state where the first wall 13a protrudes maximally toward the side of the first liquid feeding channel 7a.

It may be preferable that the area Sa be less than half the area of the first liquid feeding channel 7a in the state of having a circular shape.

An area Sb indicates the amount of change in cross-sectional area of the second liquid feeding channel 7b between a state where the second liquid feeding channel 7b enclosed by the second part 12b and the second wall 13b has a circular shape, and a state where the second wall 13b protrudes maximally toward the side of the second liquid feeding channel 7b.

It may be preferable that the area Sb be less than half the area of the second liquid feeding channel 7b in the state of having a circular shape.

The above restriction on the amount of protrusion of the first wall 13a and the second wall 13b is a restriction to prevent the first liquid feeding channel 7a and the second liquid feeding channel 7b from being unnecessarily blocked when the treatment instrument channel 5 is in a state of being expanded in diameter.

However, when use of a large-diameter treatment instrument 90 is assumed, the amount of protrusion of the walls 13 toward the side of the liquid feeding channel 7 may be restricted in accordance with the outer diameter Rx of treatment instrument 90 so that the amount of change in cross-sectional area is less than a value smaller than half the area when the channel has a circular shape (for example, 40%, 30%, etc.).

Incidentally, the endoscope 1 can be reprocessed by using a reprocessing method.

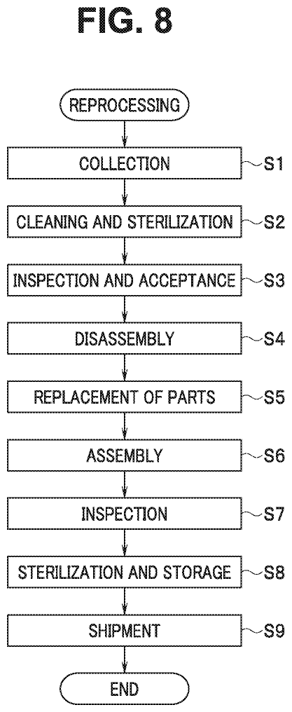

The endoscope 1 mentioned above may be discarded after a single use or may be reused a plurality of times. When the endoscope 1 is configured to be repeatedly used a plurality of times, a reprocessing method such as that shown in FIG. 8 may be required.

FIG. 8 is a flowchart showing a reprocessing method for the endoscope 1 in the first embodiment.

An operator may perform consolidation, such as collection and receipt, of a used endoscope 1 after being used for examination, and perform transportation, such as carriage, shipment, delivery, of the endoscope to a factory or the like (step S1).

Next, the operator may clean and sterilize the collected and transported used endoscope 1 (step S2).

Next, the operator may perform inspection and acceptance of the used endoscope 1 (step S3).

Thereafter, the operator may disassemble the used endoscope 1 (step S4), and replace some parts of the used endoscope 1 with new parts (step S5).

After step S5, the operator assembles a new endoscope 1 (step S6).

In some examples, step S6 can include adding an identifier indicating that the apparatus has been modified from its original state, for example, adding a label or another marking designating the apparatus as a reprocessed, refurbished, or remanufactured product.

After step S6, the operator may sequentially perform inspection (step S7), sterilization and storage (step S8), and shipment (step S9) of the new endoscope 1.

The endoscope 1 according to the present embodiment has a shape (structure) in which the first part 12a and the second part 12b protrude from the third part 12c in a cross-sectional view perpendicular to the longitudinal direction A of the endoscope multi-lumen tube 11. The outer contours 12a1 and 12a2 and the second outer contours 12b1 and 12b2 each form a recessed shape. Therefore, in step S5, there is an advantage of making it easier to perform positioning and rearrangement of internal components other than the endoscope multi-lumen tube 11.

The reprocessing of the endoscope 1 is achieved by executing the above-mentioned steps S1 to S9. Although specific sequence terminology is used above, one or a plurality of steps of the present method may be executed out of sequence depending on the situation. It should be understood that, unless a specific sequence is claimed, reliance should not be placed on the present specification requiring a specific sequence when evaluating the meaning or scope of the claims.

According to the first embodiment, the walls 13 partitioning the treatment instrument channel 5 and the liquid feeding channel 7 of the endoscope multi-lumen tube 11, which is provided with the treatment instrument channel 5 and the liquid feeding channel 7, are formed of a deformable material. Therefore, when inserting the treatment instrument 90 through the treatment instrument channel 5, the inner diameter of the treatment instrument channel 5 can be secured, and when feeding liquid through the liquid feeding channel 7, the inner diameter of the liquid feeding channel 7 can be secured.

Thus, the endoscope multi-lumen tube 11 and the endoscope 1 can be provided that can increase the number of types of applicable treatment instruments 90 and avoid a decline in fluid supply performance without increase in diameter of the insertion portion 2.

The tube 12 is configured such that, in a cross-section perpendicular to the longitudinal direction A, the first part 12a in which the first liquid feeding channel 7a is provided, and the second part 12b in which the second liquid feeding channel 7b is provided each protrude relative to the third part 12c in which the treatment instrument channel 5 is provided. Furthermore, the treatment instrument channel 5 and the first liquid feeding channel 7a are partitioned by the first wall 13a, and the treatment instrument channel 5 and the second liquid feeding channel 7b are partitioned by the second wall 13b. By adopting this configuration in the endoscope multi-lumen tube 11, it becomes easier to secure, within the treatment instrument channel 5, a space through which to insert the treatment instrument 90 even during liquid feeding, and it is possible to avoid unintended decline in the amount of liquid feeding caused by the treatment instrument 90 contacting at least one of the first wall 13a and the second wall 13b.

### Second Embodiment

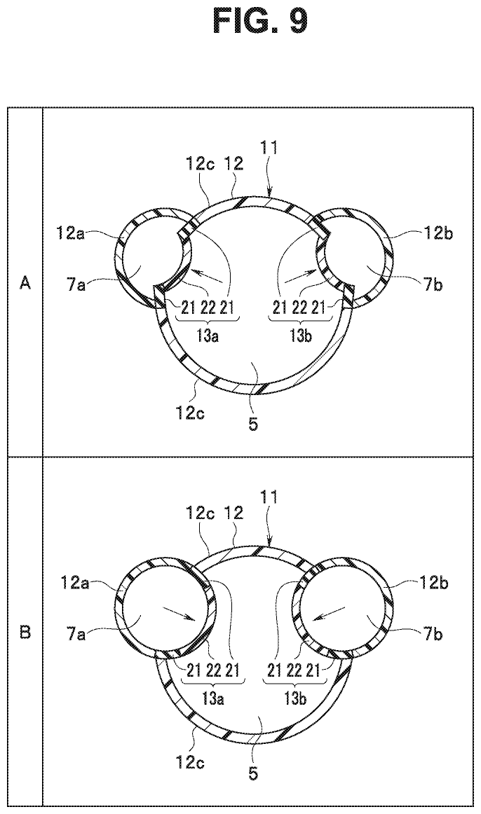

FIG. 9 is a diagram for illustrating a configuration and operation of the first wall 13a and the second wall 13b partitioning the interior of the tube 12 in the endoscope multi-lumen tube 11 of a second embodiment of the present disclosure.

In the second embodiment, the same parts as in the first embodiment are assigned the same reference numerals, and descriptions thereof will be omitted as appropriate. In the second embodiment, points differing from the first embodiment will be mainly described.

Column A of FIG. 9 shows how the first wall 13a and second wall 13b change when the treatment instrument 90 is inserted through the treatment instrument channel 5.

Column B of FIG. 9 shows how the first wall 13a and the second wall 13b change when fluid is injected into the first liquid feeding channel 7a and the second liquid feeding channel 7b.

In the first embodiment, the walls 13 (first wall 13a and second wall 13b) are configured of a material having, for example, a Shore hardness of 10. However, if the entire wall 13 is configured of a soft material, a large amount of energy is required to restore the deformed wall 13 to the original shape. Therefore, in the present embodiment, the walls 13 are configured as follows.

The wall 13 (first wall 13a and second wall 13b) each include a flexible part 21 and a rigid part 22 in cross-section, wherein the flexible part 21 is deformable.

In cross-section, for example, both end sides of the wall 13 connected to the tube 12 constitute the flexible part 21, and the part sandwiched between the flexible parts 21 at both end side constitutes the rigid part 22.

The wall 13 includes the flexible part 21 configured of a material having a Shore hardness of 30 or less, and the rigid part 22 configured of a material having a Shore hardness of 55 or more. The Shore hardness of the material configuring the flexible part 21 may be 10 or more and 30 or less. The Shore hardness of the material configuring the rigid part 22 may be 55 or more and 75 or less.

To give a specific numerical example, the flexible part 21 is configured of a material having a Shore hardness of 10, and the rigid part 22 is configured of a material having a Shore hardness of 65.

Regarding the first wall 13a, the total area of the flexible parts 21 at both end sides in a cross-section perpendicular to the longitudinal direction A may be approximately one-third of the area of the first wall 13a.

Similarly, regarding the second wall 13b, the total area of the flexible parts 21 at both end sides in a cross-section perpendicular to the longitudinal direction A may be approximately one-third of the area of the second wall 13b.

According to the second embodiment, effects substantially the same as those of the first embodiment are yielded.

Furthermore, according to the second embodiment, the walls 13 (first wall 13a and second wall 13b) are each configured to have the flexible part 21 and the rigid part 22. This lessens the amount of the flexible part compared to the walls 13 of the first embodiment, thereby reducing the energy required for deformation. Consequently, the shape of the walls 13 after deformation can be more easily restored to the original shape.

Incidentally, if the walls 13 are configured solely of a flexible part, or configured with an increased amount of the flexible part, controlling the movable range of the walls 13 becomes difficult. For example, a Shore hardness of 10 is roughly the same as the Shore hardness of balloons that are sometimes used in endoscopy, but balloons with a Shore hardness of 10 are difficult to control in shape during expansion.

In contrast, the walls 13 of the second embodiment each partially include the rigid part 22, and the shape of the rigid part 22 basically does not change even when the shape of the flexible part 21 changes, which makes shape control (control of the movable range) of the walls 13 more reliable. This facilitates configuring the wall 13 such that the maximum protrusion length of the walls 13 toward the side of the treatment instrument channel 5 is less than 25% of the maximum diameter R1 of the treatment instrument channel 5.

### Related Art

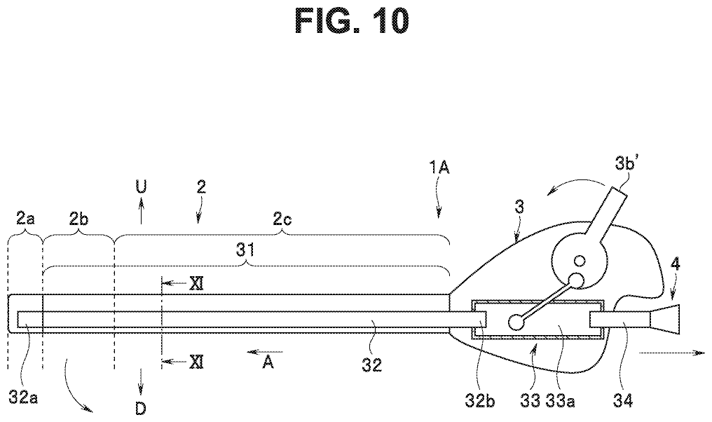

FIGS. 10 to 14 illustrate a related art of the above embodiments. In the related art, the same parts as in the above embodiments are assigned the same reference numerals, and descriptions thereof will be omitted as appropriate. In the related embodiment, points differing from the first embodiment will be mainly described.

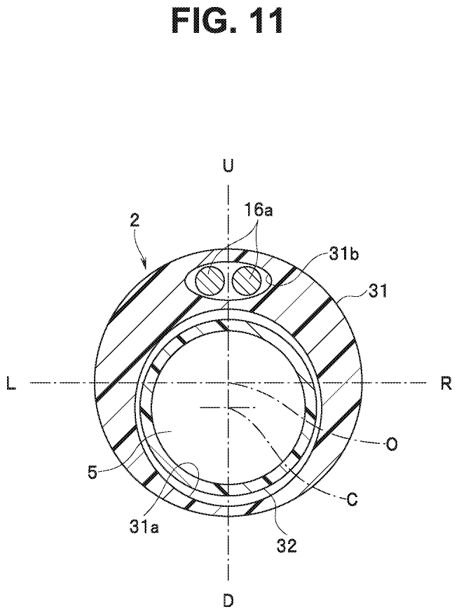

FIG. 10 is a view showing a configuration of an endoscope 1A of the related art. FIG. 11 is a cross-sectional view of the endoscope 1A of the related art along line X-X shown in FIG. 10.

The endoscope 1A includes the insertion portion 2, the operation portion 3, and the universal cable 4. In the example configuration of FIG. 10, instead of the bending operation knob 3b of the above embodiments, a bending operation lever 3b′ is arranged on the operation portion 3.

The insertion portion 2 includes, sequentially from the distal end side toward the proximal end side, the distal end portion 2a, the bending portion 2b, and the flexible tube portion 2c.

The bending portion 2b and the flexible tube portion 2c of the insertion portion 2 are configured as a multi-lumen tube 31 including a plurality of lumens 31a and 31b, and the like. The distal end portion 2a is coupled integrally with the distal end side of the multi-lumen tube 31.

In a cross-section perpendicular to the longitudinal direction A shown in FIG. 11, relative to the center O of the insertion portion 2, the lumen 31a is arranged, for example, at a position offset in a downward direction D in the bending direction, and the lumen 31b is arranged, for example, at a position offset in an upward direction U in the bending direction.

The treatment instrument channel tube 32 is inserted through the lumen 31a. The interior of the treatment instrument channel tube 32 serves as the treatment instrument channel 5. For example, the signal cable 16a and the like are inserted through the lumen 31b.

A distal end side part 32a of the treatment instrument channel tube 32 is fixed to the distal end portion 2a. A proximal end side part 32b of the treatment instrument channel tube 32 is disposed in the interior of the operation portion 3.

A link mechanism 33, which converts the rotational motion of the bending operation lever 3b′ into linear motion, is disposed in the interior of the operation portion 3. The proximal end side part 32b of the treatment instrument channel tube 32 is connected to the distal end side of the link mechanism 33.

The proximal end side of the link mechanism 33 is connected to a suction tube 34. An interior 33a of the link mechanism 33 establishes communication between the channel in the interior of the treatment instrument channel tube 32 and the channel in the interior of the suction tube 34.

As shown in FIG. 11, in a cross-section perpendicular to the longitudinal direction A, a center C of the treatment instrument channel tube 32 is arranged at a position offset from the center O of the insertion portion 2, for example, in the downward direction D. It should be noted that the center C may be arranged at a position offset from the center O in the upward direction U, instead of being arranged at a position offset in the downward direction D.

When it is only required to perform an RL bending, the center C may be arranged at a position offset from the center O in a right direction R or a left direction L in the bending direction.

When the bending operation lever 3b′ is pivoted, the link mechanism 33 converts the rotational motion into linear motion, causing the treatment instrument channel tube 32 and the suction tube 34 to move in the longitudinal direction of the insertion portion 2.

For example, when the bending operation lever 3b′ is pivoted in a counterclockwise direction in FIG. 10, the treatment instrument channel tube 32 and the suction tube 34 move in the proximal end direction. Consequently, the distal end side part 32a of the treatment instrument channel tube 32, to which the distal end portion 2a of the endoscope 1A is fixed, is drawn in the proximal end direction, causing the bending portion 2b to bend in the downward direction D. This orients the distal end portion 2a in the downward direction D.

When the bending operation lever 3b′ is pivoted in a clockwise direction in FIG. 10, the treatment instrument channel tube 32 and the suction tube 34 move in the distal end direction. Consequently, the distal end portion 2a of the endoscope 1A is pressed in the distal end direction, causing the bending portion 2b to bend in the upward direction U. This orients the distal end portion 2a in the upward direction U.

In this way, the treatment instrument channel tube 32 is configured to function as a bending operation wire. Consequently, the bending operation wire for bending in the up and down two directions can be omitted, and furthermore, there is no need to provide the multi-lumen tube 31 with a lumen through which to insert the bending operation wire. Since a lumen in which to arrange a bending operation wire is unnecessary, the treatment instrument channel tube 32 having a larger inner diameter can be adopted without increasing the diameter of the insertion portion 2, enabling the treatment instrument 90 having a larger diameter to be inserted through the treatment instrument channel 5.

Thus, it is possible to increase the number of types of applicable treatment instrument 90 without increase in diameter of the insertion portion 2, thereby providing the endoscope 1A with improved treatment performance.

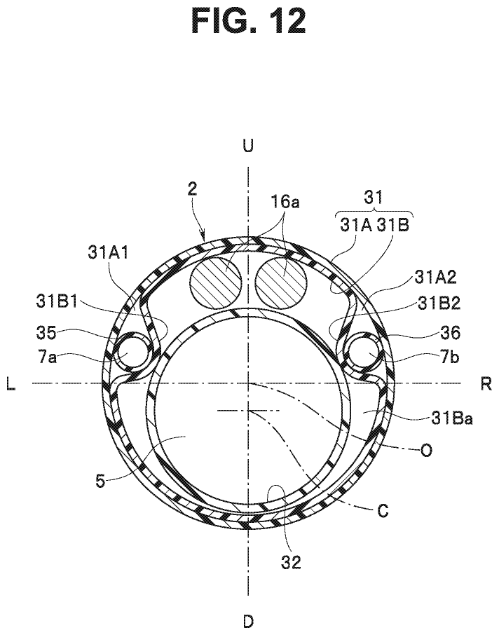

FIG. 12 is a cross-sectional view of the insertion portion 2 of the endoscope 1A in a first modified example of the related art, taken perpendicular to the longitudinal direction.

The multi-lumen tube 31 of the first modified example is configured of a two-layer structure consisting of an outer layer tube 31A and an inner layer tube 31B. The outer layer tube 31A is formed of a material such as, for example, PEBA (polyether block amide). The inner layer tube 31B is formed of a material such as, for example, PTFE (polytetrafluoroethylene).

The outer layer tube 31A also serves as, for example, an outer sheath of the insertion portion 2. It should be noted that a braid (a tubular member formed by braiding strands of metal, resin, or the like) may be provided on an inner surface of the outer layer tube 31A to increase the strength of the multi-lumen tube 31.

The inner layer tube 31B is provided on an inner circumferential side of the outer layer tube 31A basically along an inner circumferential surface of the outer layer tube 31A. However, the inner layer tube 31B includes, in parts in a circumferential direction in a cross-section perpendicular to the longitudinal direction A, a first protruding part 31B1 and a second protruding part 31B2 that are distanced from the inner circumferential side of the outer layer tube 31A to extend in an inner diameter direction.

A lumen 31A1 is formed between the outer layer tube 31A and the first protruding part 31B1. A first liquid feeding channel tube 35 is inserted through the lumen 31A1. The interior of the first liquid feeding channel tube 35 serves as the first liquid feeding channel 7a.

A lumen 31A2 is formed between the outer layer tube 31A and the second protruding part 31B2. The second liquid feeding channel tube 36 is inserted through the lumen 31A2. The interior of the second liquid feeding channel tube 36 serves as the second liquid feeding channel 7b.

A lumen 31Ba is formed inside the inner layer tube 31B. The treatment instrument channel tube 32 is inserted through the lumen 31Ba, and furthermore, internal components other than the treatment instrument channel tube 32 are arranged in the lumen 31Ba. Examples of the internal components other than the treatment instrument channel tube 32 that are inserted through the lumen 31Ba include a plurality of signal cables 16a and the like. The interior of the treatment instrument channel tube 32 serves as the treatment instrument channel 5, as mentioned above.

The center C of the treatment instrument channel tube 32 is arranged at a position offset from the center O of the insertion portion 2, for example, in the downward direction D, and the treatment instrument channel tube 32 functions as a bending operation wire, as mentioned above.

At this time, the first protruding part 31B1 and the second protruding part 31B2 constrain the movement of the treatment instrument channel tube 32 within the lumen 31Ba (movement within a plane perpendicular to the longitudinal direction A). This holds the positional relationship where the center C of the treatment instrument channel tube 32 is offset from the center O of the insertion portion 2 in the downward direction D even when the bending portion 2b is bent.

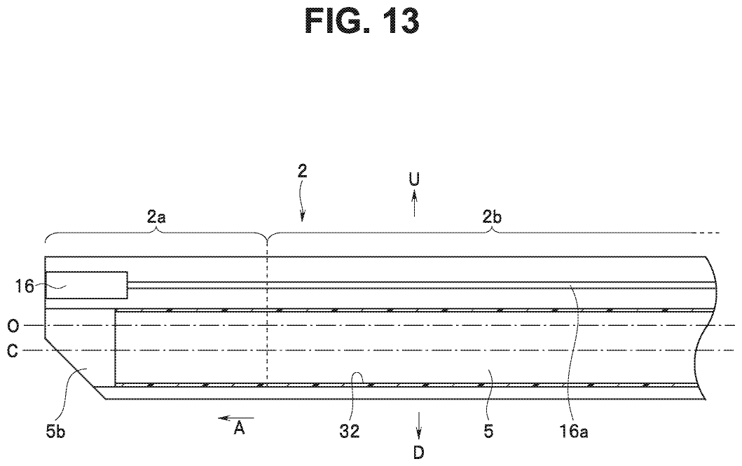

FIG. 13 is a view showing arrangement of the treatment instrument channel tube 32 in the insertion portion 2 of the endoscope 1A in a second modified example of the related art.

In a typical endoscope, a bending operation wire is arranged within the insertion portion 2 unlike the endoscope 1A of the related art mentioned above. Therefore, in a typical endoscope, the treatment instrument channel tube 32 is arranged at a position near the center O of the insertion portion 2 within the bending portion 2b and the flexible tube portion 2c. This is to arrange the treatment instrument channel tube 32 while avoiding the bending operation wire for the upward direction U and the bending operation wire for the downward direction D.

On the other hand, in a typical endoscope, the treatment instrument channel tube 32 is flexed in the downward direction D within the distal end portion 2a, is deviated in the downward direction D relative to the center O, and is guided to the distal end side opening 5b of the treatment instrument channel, in order to avoid the image pickup module 16 arranged, for example, on the side of the upward direction U of the distal end portion 2a.

In contrast to such a typical endoscope, the endoscope 1A of the related art is not provided with a bending operation wire for the upward direction U or a bending operation wire for the downward direction D, as mentioned above. As shown in FIG. 11, the center C of the treatment instrument channel tube 32 is arranged, for example, at a position offset from the center O of the insertion portion 2 in the downward direction D.

Therefore, as shown in FIG. 13, the treatment instrument channel tube 32 is arranged in a straight line within the insertion portion 2 including the flexible tube portion 2c, the bending portion 2b, and the distal end portion 2a, and further communicates with the distal end side opening 5b of the treatment instrument channel in a straight line.

This simplifies the structure of the insertion portion 2 and improves the assemblability thereof. Furthermore, since the treatment instrument channel tube 32 is arranged in a straight line, the insertability of the treatment instrument 90 is also enhanced.

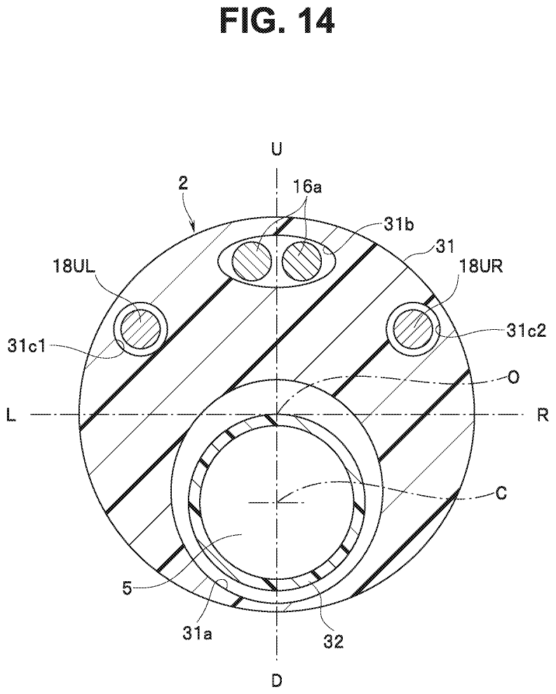

FIG. 14 is a cross-sectional view of the insertion portion 2 of the endoscope 1A in a third modified example of the related art, taken perpendicular to the longitudinal direction A.

In the configuration of FIGS. 10 to 13, the treatment instrument channel tube 32 is movable in the distal end direction and the proximal end direction, thereby enabling the bending portion 2b to bend in two directions of the upward direction U and the downward direction D (or two directions of the right direction R and the left direction L).

There are cases where it is desirable for the endoscope to be bendable in four directions: the upward direction U, the downward direction D, the right direction R, and the left direction L.

Therefore, a configuration as shown in FIG. 14 may be adopted.

The multi-lumen tube 31 further includes lumens 31c1 and 31c2 in addition to the lumens 31a and 31b shown in FIG. 11.

As in FIG. 11, the treatment instrument channel tube 32 is inserted through the lumen 31a, and the signal cable 16a and the like are inserted through the lumen 31b.

In a cross-section perpendicular to the longitudinal direction A shown in FIG. 14, relative to the center O of the insertion portion 2, the lumen 31c1 is arranged at a position offset in the UL direction (the upward direction U and left direction L in the bending direction), and the lumen 31c2 is arranged at a position offset in the UR direction (upward direction U and right direction R in the bending direction).

A bending operation wire 18UL is inserted through the lumen 31c1, and a bending operation wire 18UR is inserted through the lumen 31c2.

In the configuration of the third modified example of the related art, the bending operation is performed as follows, for example.

Moving (pulling) the treatment instrument channel tube 32 in the proximal end direction causes the bending portion 2b to bend in the downward direction D.

By simultaneously pulling the bending operation wire 18UL and the bending operation wire 18UR by the same amount, the bending portion 2b bends in the upward direction.

By pulling the bending operation wire 18UR, and also moving the treatment instrument channel tube 32 in the proximal end direction by adjusting the amount of movement (pulling with an adjusted pulling amount), the bending portion 2b bends in the right direction R. This is due to the following reason. If only the bending operation wire 18UR is pulled, the bending portion 2b not only bends in the right direction R but also bend in the upward direction U. Therefore, to suppress the bending in the upward direction U, the treatment instrument channel tube 32 is also pulled with an adjusted pulling amount.

Pulling the bending operation wire 18UL, and also moving the treatment instrument channel tube 32 in the proximal end direction by adjusting the amount of movement (pulling with an adjusted pulling amount), the bending portion 2b bends in the left direction L. This is due to the following reason. If only the bending operation wire 18UL is pulled, the bending portion 2b not only bends in the left direction L but also bends in the upward direction U. Therefore, to suppress the bending in the upward direction U, the treatment instrument channel tube 32 is also pulled with an adjusted pulling amount.

A known technique using three bending operation wires to perform four-directional bending can be appropriately utilized for the configuration of FIG. 14 in which four-directional bending is performed by pulling a total of three elements: the treatment instrument channel tube 32 and the bending operation wires 18UL and 18UR.

It should be noted that in the example shown in FIG. 14, two bending operation wires 18UL and 18UR are added to enable four-directional bending operation, but no limitation is placed on this configuration.

For example, a configuration may be adopted in which three bending operation wires are added respectively in the upward direction U, the right direction R, and the left direction L in the bending direction, as viewed from the center O. In this case, pulling a bending operation wire for the upward direction U causes the bending portion 2b to bend in the upward direction U, pulling a bending operation wire for the right direction R causes the bending portion 2b to bend in the right direction R, pulling a bending operation wire for the left direction L causes the bending portion 2b to bend in the left direction L, and pulling the treatment instrument channel tube 32 causes the bending portion 2b to bend in the downward direction D.

According to the configuration of the third modified example of the related art shown in FIG. 14, it becomes possible to bend the bending portion 2b in four directions.

It should be noted that a configuration may be adopted in which the bending operation wire for the upward direction U is added to the endoscope 1A capable of bending in two directions: the upward direction U and the downward direction D. In this case, pulling the bending operation wire for the upward direction U causes the bending portion 2b to bend in the upward direction U, and pulling the treatment instrument channel tube 32 causes the bending portion 2b to bend in the downward direction D. Adopting this configuration eliminates the need to press and move the treatment instrument channel tube 32 in the distal end direction when the bending portion 2b is bent in the upward direction U. This prevents the treatment instrument channel tube 32 from flexing due to the pressing.

It should be noted that the present disclosure is not limited to the embodiments mentioned above on an as-is basis. In the implementation stage, the present disclosure can be embodied by modifying constituent elements within the scope that does not depart from the gist of the invention. Various aspects of the invention can be formed by appropriately combining a plurality of constituent elements disclosed in the above embodiments. For example, some constituent elements may be omitted from all constituent elements disclosed in the embodiments. Furthermore, constituent elements of different embodiments may be appropriately combined. Thus, it is needless to say that various modifications and applications are possible within the scope that does not depart from the gist of the invention.

The present application claims priority from U.S. Patent Application No. 63/550,634, filed in the United States of America on Feb. 7, 2024. The content disclosed in the above priority application is incorporated by reference into the specification, claims, and drawings of the present application.

What is claimed is:

1. An endoscope multi-lumen tube comprising:
a tube arranged within an insertion portion of an endoscope in a longitudinal direction;
a deformable wall provided along the longitudinal direction and partitioning an interior of the tube;
a first channel formed on one portion of the interior of the tube partitioned by the wall; and
a second channel formed on another portion of the interior of the tube partitioned by the wall, wherein
in a cross-section perpendicular to the longitudinal direction, the tube is configured such that:
a side of the first channel protrudes toward one side relative to the wall;
a side of the second channel protrudes toward another side relative to the wall; and
outer contours of two parts connected to the wall each form a recessed shape.

2. The endoscope multi-lumen tube according to claim 1, wherein a distance between both ends of the wall in the cross-section is smaller than a maximum diameter of the first channel and smaller than a maximum diameter of the second channel.

3. The endoscope multi-lumen tube according to claim 1, wherein the wall watertightly separates the first channel from the second channel.

4. The endoscope multi-lumen tube according to claim 1, wherein
the first channel is a treatment instrument channel configured to allow insertion of a treatment instrument therethrough,
the second channel is a liquid feeding channel configured to allow a fluid to be injected thereinto, and
the wall protrudes toward the side of the first channel by a pressure of the fluid when the fluid is injected into the second channel.

5. The endoscope multi-lumen tube according to claim 4, wherein, by suctioning the fluid within the second channel after stopping injection of the fluid into the second channel, a negative pressure of the suctioned fluid causes the wall to protrude toward the side of the second channel, thereby expanding a diameter of the first channel.

6. The endoscope multi-lumen tube according to claim 4, wherein, when the treatment instrument is inserted through the first channel and presses against the wall, the wall protrudes toward the side of the second channel.

7. The endoscope multi-lumen tube according to claim 6, wherein the wall is configured such that a maximum protrusion length toward the side of the first channel when the fluid is injected into the second channel is less than 25% of a maximum diameter of the first channel.

8. The endoscope multi-lumen tube according to claim 7, wherein
the tube is configured of a material having a Shore hardness of 55 or more, and
the wall is configured of a material having a Shore hardness of 30 or less.

9. The endoscope multi-lumen tube according to claim 1, wherein the wall includes a flexible part and a rigid part in the cross-section, and the flexible part is deformable.

10. The endoscope multi-lumen tube according to claim 9, wherein in the cross-section, both end sides of the wall connected to the tube constitute the flexible parts, and a part sandwiched between the flexible parts at the both end side constitutes the rigid part.

11. The endoscope multi-lumen tube according to claim 9, wherein the wall includes the flexible part configured of a material having a Shore hardness of 30 or less, and the rigid part configured of a material having a Shore hardness of 55 or more.

12. The endoscope multi-lumen tube according to claim 1, wherein, with the outer contours each forming the recessed shape, the tube can prevent internal components other than the tube within the insertion portion from being displaced within the insertion portion.

13. The endoscope multi-lumen tube according to claim 1, further comprising:
a deformable second wall provided along the longitudinal direction and partitioning the interior of the tube;
the first channel arranged in one portion of the interior of the tube partitioned by the second wall; and
a third channel formed on another portion of the interior of the tube partitioned by the second wall, wherein
in a cross-section perpendicular to the longitudinal direction, the tube is configured such that:
a side of the third channel protrudes toward a side different from the side of the first channel relative to the second wall, and
second outer contours of two parts connected to the second wall each form a recessed shape.

14. The endoscope multi-lumen tube according to claim 13, wherein a distance between both ends of the second wall in the cross-section is smaller than a maximum diameter of the first channel and smaller than a maximum diameter of the third channel.

15. The endoscope multi-lumen tube according to claim 13, wherein, with the second outer contours each forming the recessed shape, the tube can prevent internal components other than the tube within the insertion portion from being displaced within the insertion portion.

16. An endoscope comprising:
an insertion portion; and
an endoscope multi-lumen tube,
the endoscope multi-lumen tube comprising:
a tube arranged within the insertion portion of an endoscope in a longitudinal direction;
a deformable wall provided along the longitudinal direction and partitioning an interior of the tube;
a first channel formed on one portion of the interior of the tube partitioned by the wall; and
a second channel formed on another portion of the interior of the tube partitioned by the wall, wherein
in a cross-section perpendicular to the longitudinal direction, the tube is configured such that:
a side of the first channel protrudes toward one side relative to the wall;
a side of the second channel protrudes toward another side relative to the wall; and
outer contours of two parts connected to the wall each form a recessed shape.

17. The endoscope according to claim 16, wherein a distance between both ends of the wall in the cross-section is smaller than a maximum diameter of the first channel and smaller than a maximum diameter of the second channel.

18. The endoscope according to claim 16, wherein the wall watertightly separates the first channel from the second channel.

19. The endoscope according to claim 16, wherein
the first channel is a treatment instrument channel configured to allow insertion of a treatment instrument therethrough,
the second channel is a liquid feeding channel configured to allow a fluid to be injected thereinto, and
the wall protrudes toward the side of the first channel by a pressure of the fluid when the fluid is injected into the second channel.

20. The endoscope according to claim 16, wherein the wall includes a flexible part and a rigid part in the cross-section, and the flexible part is deformable.
